(19) United States
(12) Patent Application Publication
(10) Pub. No.: US 2017/0281469 A1
(43) Pub. Date: Oct. 5, 2017

(54) Systems and Methods for Compounding Injectable Therapeutic Agents

(71) Applicant: Pentec Health, Inc., Glen Mills, PA (US)

(72) Inventors: Steven A. D'Amico, Glen Mills, PA (US); Michael Abens, Glen Mills, PA (US); Anthony Bonelli, Glen Mills, PA (US)

(21) Appl. No.: 15/478,621

(22) Filed: Apr. 4, 2017

Related U.S. Application Data
(60) Provisional application No. 62/318,370, filed on Apr. 5, 2016.

(51) Int. Cl. A61J 1/20; A61K 49/00; A61K 31/5513; A61K 31/485; A61K 31/137; A61K 31/704; A61K 31/7036; A61K 33/04; A61K 31/727; A61K 31/135; A61K 38/11; A61J 1/14; A61M 5/178; A61J 1/10; A61K 9/00

(52) U.S. Cl. CPC A61J 1/2096; A61K 9/0019; A61K 49/0002; A61K 31/5513; A61K 31/485; A61K 31/137; A61K 31/704; A61K 31/7036; A61K 33/04; A61K 31/727; A61K 31/135; A61K 38/11; A61J 1/2089; A61J 1/1406; A61J 1/201; A61M 5/178; A61J 1/10

(57) ABSTRACT

Methods for compounding a therapeutic agent for intravenous administration. The method comprise providing a syringe or vial comprising the agent dissolved or pre-dissolved in a carrier at a dose or concentration that is therapeutically effective for a particular patient but is not commercially available at the therapeutically effective dose or concentration or is on the U.S. FDA drug shortage list, and adding the therapeutic agent dissolved in the carrier to an intravenous solution (diluent). The methods exclude reconstituting multiple solid forms of the commercially available agent and combining the reconstituted preparations together, for example, according to traditional compounding procedures at point-of-care pharmacies.

### CROSS REFERENCE TO RELATED APPLICATIONS

This application claims priority to U.S. Provisional App. No. 62/318,370, filed on Apr. 5, 2016, which is incorporated by reference herein in its entirety.

### FIELD OF THE INVENTION

The invention relates generally to the field of pharmaceutical compounding. More particularly, the invention relates to systems and methods for compounding injectable pharmaceutical agents at doses and concentrations that are not commercially available or are listed on the FDA drug shortage list. The systems and methods facilitate the manufacture of ready-to-use therapeutic products for patient administration. Pre-compounding agents in this way eliminates many regulation-mandated or quality control (QC)-mandated steps that make hospital compounding processes cumbersome and wasteful, and substantially reduces human error attendant to hospital-based compounding.

### BACKGROUND OF THE DISCLOSURE

Various publications, including patents, published applications, accession numbers, technical articles and scholarly articles are cited throughout the specification. Each of these cited publications is incorporated by reference, in its entirety and for all purposes, in this document.

Many pharmaceuticals are manufactured and sold in a single particular amount, per what was U.S. FDA approved. Nevertheless, this as-manufactured amount is not necessarily the dosage, or amenable to the volume or concentration that a given patient will require, particularly for pharmaceuticals that are administered via injection or infusion. To attain the correct dosage, volume, and/or concentration, such pharmaceuticals are typically compounded in the hospital pharmacy, where the amount as-manufactured is reconstituted and/or mixed with a diluent at the desired dosage, volume, and/or concentration.

Point-of-care facility compounding protocols are governed by the U.S. Pharmacopeial (USP) Convention Chapter 797 (“Pharmaceutical Compounding—Sterile Preparations”). Chapter 797 sets forth minimum practices and quality standards for compounding pharmaceuticals, and are in place to prevent harm from microbial contamination, endotoxin contamination, variability from the intended strength, chemical and physical contamination, and the use of ingredients of inappropriate quality.

A typical point-of-care facility compounding process includes three stages: staging, compounding, and verification/checking. Staging includes obtaining all of the materials that will be needed to compound the agent, including the agent, diluents, tools, and disinfectant materials. Compounding includes the steps to reconstitute the agent, and combine as needed and mix with a diluent to attain the desired dose or concentration. Verification/checking includes the steps that a second individual (e.g., pharmacist) must carry out to verify that the pharmaceutical was correctly compounded.

An example of the traditional process used for compounding the antibiotic vancomycin is shown in FIG. 1. As shown, staging includes obtaining two needles, two syringes, two vials of vancomycin, a bottle of sterile water for injection (SWFI), a bag of intravenous fluid diluent, and sterile alcohol pads. The compounding process then involves twenty three steps, several of which include drawing liquid volumes into the syringe, relying on the precision of eye-balling syringe graduation lines to attain the correct volume each time. The verification/checking process then involves inspection of the tools used to compound the pharmaceutical, to ensure that the correct volumes were used. Syringe pull-back is used to verify how much of the agent or the diluent was added to the infusion bag.

Precision is paramount. For many pharmaceuticals, the error tolerance window for the correct dose and concentration is very narrow, with small deviations to the downside affecting efficacy and with small deviations to the upside potentially causing untoward effects in the patient. Moreover, in many cases, for example, where a drug is administered several times per day, the error effects are cumulative. Nevertheless, the error rate is high, with some studies reporting 10% error rate (as high as 37% error for parenteral nutrition solutions) (Flynn E A et al. (1997) Am. J. Health Sys. Pharm. 15:904-15).

It has been suggested that automation can be employed in compounding in order to reduce human error and save cost. Nevertheless, automated systems are complex and themselves costly, and subject to breakdown. Thus, there remains a need to improve hospital compounding.

### BRIEF SUMMARY OF THE DISCLOSURE

A method for compounding a therapeutic agent for intravenous administration comprises providing a syringe containing the therapeutic agent dissolved in a carrier at a dose or concentration that is therapeutically effective for a particular patient but not commercially available at the therapeutically effective dose or concentration or is on the U.S. FDA drug shortage list, disinfecting the syringe and disinfecting a port on a container containing a diluent, connecting the disinfected syringe to the disinfected port, injecting the therapeutic agent dissolved in the carrier into the diluent via the port, and verifying that the syringe is depleted of the therapeutic agent dissolved in the carrier. The disinfected syringe may be connected to the disinfected port via a needle or luer lock. According to the method, the therapeutic agent is not dissolved in the carrier by drawing the carrier into a first syringe, injecting the carrier from the first syringe into a vial containing the agent and mixing the carrier with the agent to obtain a preparation of the agent, removing the preparation of the agent from the vial, and combining the preparation of the agent with another preparation of the agent.

A method for compounding a therapeutic agent for intravenous administration comprises providing a vial comprising a puncturable closure and containing the therapeutic agent dissolved in a carrier at a dose or concentration that is therapeutically effective for a particular patient but not commercially available at the therapeutically effective dose or concentration or is on the U.S. FDA drug shortage list, disinfecting the closure and optionally disinfecting a port comprising a spike and connected to a container containing a diluent, connecting the vial to the port such that the spike punctures the closure, adding the therapeutic agent dissolved in the carrier to the diluent through the port, and verifying that the vial is depleted of the therapeutic agent dissolved in the carrier. According to the method, the therapeutic agent is not dissolved in the carrier by drawing the carrier into a syringe, injecting the carrier from the syringe into the vial containing the agent and mixing the carrier with the agent to obtain a preparation of the agent; the therapeutic agent is not dissolved in the carrier by drawing the carrier into a first syringe, injecting the carrier from the syringe into the vial containing the agent in powder form and mixing the carrier with the agent to obtain a preparation of the agent, removing the preparation of the agent from the vial, and combining the preparation of the agent with another preparation of the agent; no diluent is removed from the container prior to allowing the therapeutic agent dissolved in the carrier to flow into the diluent via the port; and the volume of the agent dissolved in the carrier is less than 10% of the volume of the diluent in the container. The vial containing the therapeutic agent dissolved in the carrier preferably comprises a ratio of carrier volume to empty space of about 1 to about 1 or greater, about 1 to about 2 or greater, or about 1 to about 5 or greater.

A method for compounding a therapeutic agent for intravenous administration comprises providing a vial comprising a puncturable closure and containing the therapeutic agent dissolved in a carrier at a dose or concentration that is therapeutically effective for a particular patient but not commercially available at the therapeutically effective dose or concentration or is on the U.S. FDA drug shortage list, disinfecting the closure and disinfecting a port comprising a spike, connecting the vial to the disinfected port such that the spike punctures the closure, and connecting the port to a container containing a diluent, adding the therapeutic agent dissolved in the carrier to the diluent through the port, and verifying that the vial is depleted of the therapeutic agent dissolved in the carrier. According to the method, the therapeutic agent is not dissolved in the carrier by drawing the carrier into a syringe, injecting the carrier from the syringe into the vial containing the agent and mixing the carrier with the agent to obtain a preparation of the agent, the therapeutic agent is not dissolved in the carrier by drawing the carrier into a first syringe, injecting the carrier from the syringe into the vial containing the agent and mixing the carrier with the agent to obtain a preparation of the agent, removing the preparation of the agent from the vial, and combining the preparation of the agent with another preparation of the agent, no diluent is removed from the container prior to allowing the therapeutic agent dissolved in the carrier to flow into the diluent via the port, and the volume of the agent dissolved in the carrier is less than 10% of the volume of the diluent in the container. The vial containing the therapeutic agent dissolved in the carrier preferably comprises a ratio of carrier volume to empty space of about 1 to about 1 or greater, about 1 to about 2 or greater, or about 1 to about 5 or greater.

In any of these methods, the therapeutic agent may comprise one or more of a calcium channel blocker, antibiotic, contrast agent, local anesthetic, epidural agent, electrolyte supplement, opioid analgesic, non-steroidal anti-inflammatory analgesic, corticosteroid, proton pump inhibitor, h2 antagonist, anticholinergic, sympathomimetic vasopressor, catecholamine vasopressor, anticoagulant, benzodiazepine, oxytocic agent, alpha-adrenergic receptor agent, beta-adrenergic blocking agent, or anticonvulsant.

In any of these methods, the therapeutic agent may comprise one or more of hydromorphone, midazolam, morphine, norepinephrine, oxytocin, phenylephrine, ropivacaine, bupivacaine, lidocaine, vancomycin, gentamicin, atropine, betamethasone, calcium gluconate, cefazolin, dexamethasone, epinephrine, ephedrine, esmolol, fentanyl, flumazeril, glycopyrrolate, heparin, hydralazine, ketamine, labetalol, magnesium sulfate, metropropolol, xanthine, caffeine, rocuronium, sodium citrate, sodium thiosulfate, succinylcholine, vasopressin, verapamil, or vecuronium.

In any of these methods, the carrier comprises water, such as sterile water for injection. The diluent may be an isotonic fluid. The diluent may be a hypotonic fluid. The diluent may be a hypertonic fluid.

It is to be understood that both the foregoing general description and the following detailed description are exemplary, but are not restrictive, of the invention.

### BRIEF SUMMARY OF THE SEVERAL VIEWS OF THE DRAWING

The invention is best understood from the following detailed description when read in connection with the accompanying drawing and appended claims. It is emphasized that, according to common practice, the various features of the drawing are not to scale. On the contrary, the dimensions of the various features are arbitrarily expanded or reduced for clarity. Included in the drawing are the following figures:

FIG. 1 shows the multitude of provisions and steps required for a traditional process for the compounding of vancomycin at a point-of-care pharmacy facility;

FIG. 2 shows a compounding process for vancomycin according to the disclosure;

FIG. 3 shows the steps deleted from the traditional vancomycin compounding process shown in FIG. 1, when carried out according to the disclosure;

FIG. 4 shows an example of a syringe-based system, whereby the syringe includes a compounded pharmaceutical agent pre-packaged in a syringe at the desired dose and concentration, and the syringe accompanies a diluent for intravenous infusion;

FIGS. 5A and 5B show an example of a vial-based system, whereby the vial includes a compounded pharmaceutical agent pre-packaged in a vial at the desired dose and concentration, and the vial accompanies a diluent bag for intravenous infusion (FIG. 5A), with the bag including an adapter to which the vial may be connected to mix the agent with the diluent (FIG. 5B);

FIGS. 6A and 6B show an example of a vial-based system, whereby the vial includes a compounded pharmaceutical agent pre-packaged in a vial at the desired dose and concentration, and the vial accompanies a diluent bag for intravenous infusion and a separate bag adapter (FIG. 6A), with the adapter capable of being connected at one end to the bag and at the other end to the vial in order to mix the agent with the diluent (FIG. 6B); and

FIG. 7 compares a typical commercially available vial having a 13 mm size closure, in which the liquid volume of agent assumes nearly all of the vial's volume, with a 20 mm port-compatible vial that includes a liquid volume of the agent that assumes less than half of the vial's volume.

### DETAILED DESCRIPTION

The features and benefits of the disclosed system and method are illustrated and described by reference to exemplary embodiments. The disclosure also includes the drawing, in which like reference numbers refer to like elements throughout the various figures that comprise the drawing. This description of exemplary embodiments is intended to be read in connection with the accompanying drawing, which is to be considered part of the entire written description. Accordingly, the disclosure expressly should not be limited to such exemplary embodiments illustrating some possible non-limiting combination of features that may exist alone or in other combinations of features.

Various terms relating to aspects of the disclosure are used throughout the specification and claims. Such terms are to be given their ordinary meaning in the art, unless otherwise indicated. Other specifically defined terms are to be construed in a manner consistent with the definition provided in this document.

As used herein, the singular forms “a,” “an,” and “the” include plural referents unless expressly stated otherwise.

As used herein, the terms drug, pharmaceutical agent, therapeutic agent, active ingredient, medicine, and medicament are used interchangeably.

A syringe includes a barrel, a plunger or bulb, and an adapter such as a luer lock for connecting to a needle; some syringes are needle-less.

As used herein, the term “pre-dissolved in a carrier” or “pre-dissolved in the carrier” means that the therapeutic agent (etc.) was dissolved in a carrier by an outsourcing facility under Section 503(b) of the Food Drug and Cosmetic Act, 21 U.S.C. §353(b) (2013).

As used herein, the term “not commercially available” means that the dose, concentration, strength, form (liquid or powder), excipients and/or volume of a given therapeutic agent needed for a particular patient's treatment was not U.S. Food and Drug Administration (FDA) approved for marketing in the United States and/or not available in a ready to use final container.

As used herein, the term “U.S. FDA drug shortage list” refers to therapeutic agents that are determined to be in shortage under Section 506(e) of the Food Drug and Cosmetic Act, 21 U.S.C. §356(e) (2013).

The terms “subject” and “patient” are used interchangeably. A subject may be any animal, and preferably is a mammal. A mammalian subject may be a farm animal (e.g., sheep, horse, cow, pig), a companion animal (e.g., cat, dog), a rodent or laboratory animal (e.g., mouse, rat, rabbit), or a non-human primate (e.g., old world monkey, new world monkey). Human beings are highly preferred.

Many drugs are U.S. FDA-approved for and distributed in amounts that are not at the dose, concentration, form, or strength needed for patient administration. To convert the approved/distributed amount into the dose, concentration, form, or strength a patient requires, such drugs are compounded in the pharmacy of point-of-care facilities according to a cumbersome, complex, and error-prone compounding process governed by USP Chapter 797.

In accordance with the disclosure, it was observed that high throughput compounding can be carried out in order to create accurate concentrations and/or doses of the drugs in a ready-to-use format, while at the same time substantially reducing the risks attendant to point-of-care compounding under USP Chapter 797 (USP <797>). Thus, the disclosure features systems and methods for compounding or repackaging powdered drugs as a liquid form with precise volumes of a liquid carrier to produce concentrations and/or doses and/or packages of the drugs that are not commercially available at therapeutically effective doses or concentrations required for patient treatment. The medicines produced in this way are then administered to patients, typically intravenously. The systems and methods bypass many of the staging steps, drug compounding steps, and verification steps that are required for compounding at point-of-care facilities according to USP <797>.

Pharmaceutical agents suitable for use in accordance with the systems and methods of the disclosure are generally provided in solid form, such as a powder or crystalline form, though the systems and methods are not limited to the powder or crystalline form of such agents; the systems and methods may be used with agents in liquid form. The agents may be pharmaceutically acceptable salts of the base molecule. The solid or liquid form of the agent may also include, in addition to the active ingredient, pharmaceutically acceptable excipients, buffers, and other non-active ingredients formulated along with the active ingredient, as prepared by the manufacturer.

As part of the methods of the disclosure, such pharmaceutical agents are compounded and/or repackaged at a dose or concentration that is therapeutically effective for a particular patient, which therapeutically effective dose or concentration, or form, is not commercially available. In general, pharmaceutical agents are U.S. FDA approved and commercially available in certain amounts, which amounts are fixed according to the U.S. FDA's review and approval. For example, a given agent may only be marketed in a single amount, such as 5 mg of solid form material (e.g., powder) packaged in a vial. In some cases, a given agent may be marketed in a few different amounts, such as 5 mg, 10 mg, or 25 mg of solid form material (e.g., powder) packaged in a vial.

In any case, these approved and marketed amounts are not necessarily the amount that is therapeutically effective for a given patient's needs—a patient may need more or less, or may need a particular concentration. This is frequently the case, for example, because patient characteristics are highly variable (e.g., gender, size, age, medical condition being treated, co-morbidities, etc.). Moreover, in certain cases, the dose or concentration needed is more than what is available in a single vial but less than what is available in two vials, for example, when a dose of 15 mg is required of an agent that is available only in a 10 mg amount. In still other cases, the concentration administered to the patient is critical, but the amount of agent that is available, e.g., when reconstituted, is not amenable to simple mixing with the intravenous diluent solution—e.g., an amount of diluent would need to be removed from the intravenous container, then replaced with the same amount of reconstituted or liquid agent so as to ensure the concentration of agent, once mixed with the diluent, is correct for the patient (in some cases, a concentration too high might induce side effects and a concentration too low might not be effective; the concentration/tolerance window in such cases is quite narrow). Current practice for creating the dose or concentration required for a given patient is to compound the agent at the point-of-care facility, according to USP Chapter 797.

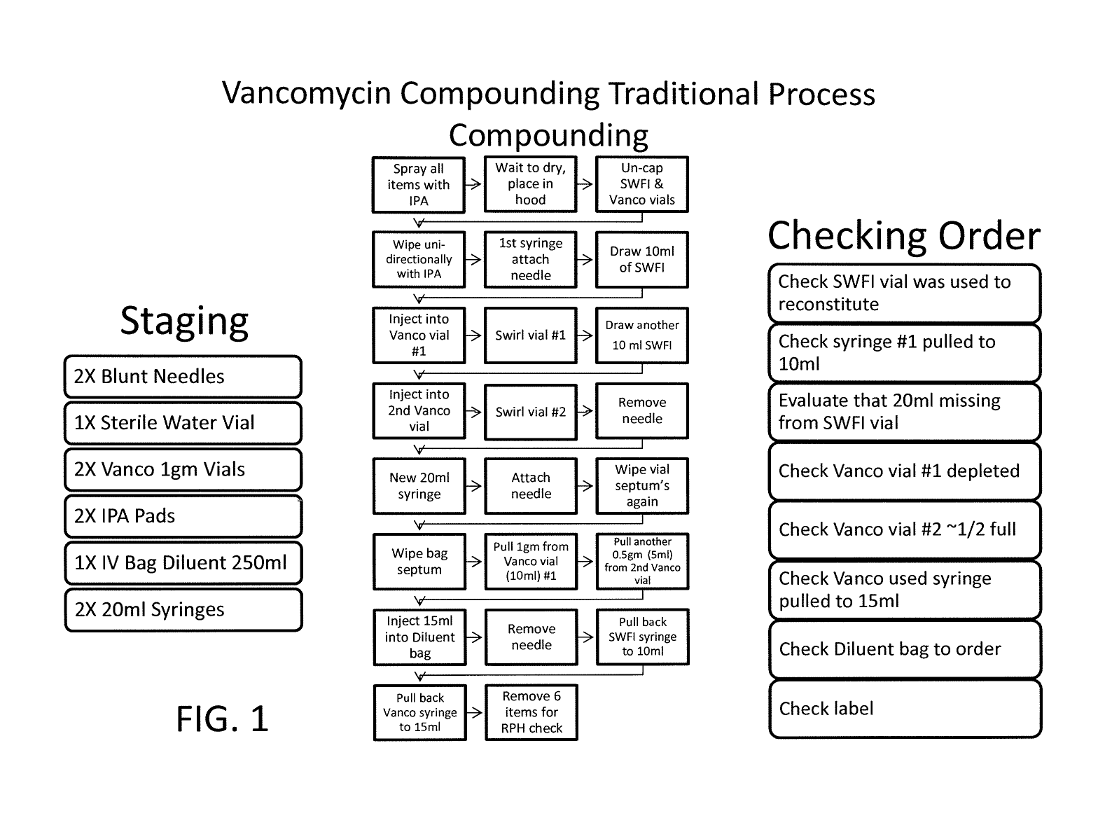

By way of example, but not of limitation, FIG. 1 illustrates the traditional point-of-care facility compounding process for vancomycin, commercially available in 1 gram amounts, though a typical required dose is 1.5 grams given intravenously up to 3 times per day. As shown, the compounding process includes staging of several pieces of equipment and materials needed to compound the drug, a twenty three stage process of compounding in which maintaining sterility throughout this process is a challenge, and an eight stage checking or verification process. As this compounding process is done by hand in the majority of cases, the process is inherently error-prone.

The systems and methods of the disclosure allow for precise preparation of patient-specific dose and concentration of intravenously administered pharmaceutical agents, such that point-of-care compounding is reduced to connecting containers of pre-fabricated, precisely measured doses and concentrations of pharmaceutical agents to a container of intravenous diluent. The containers of pre-fabricated, precisely-measured doses and concentrations of pharmaceutical agents may comprise a syringe or vial, for example, with the agent being in or reconstituted into liquid form by precisely pre-mixing the agent with a carrier and packaging the solution into the syringe or vial at a precise volume and/or concentration as appropriate for a patient's specific needs. Containers of pre-fabricated, precisely measured doses and concentrations of pharmaceutical agents are preferably prepared using automated manufacture techniques and equipment under aseptic conditions.

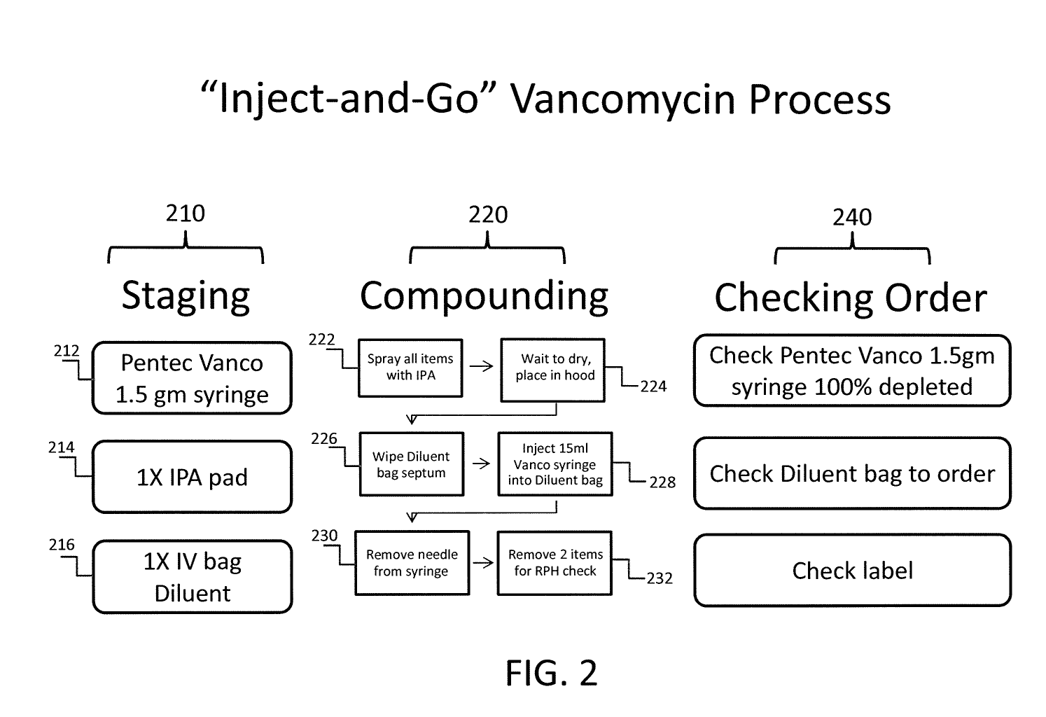

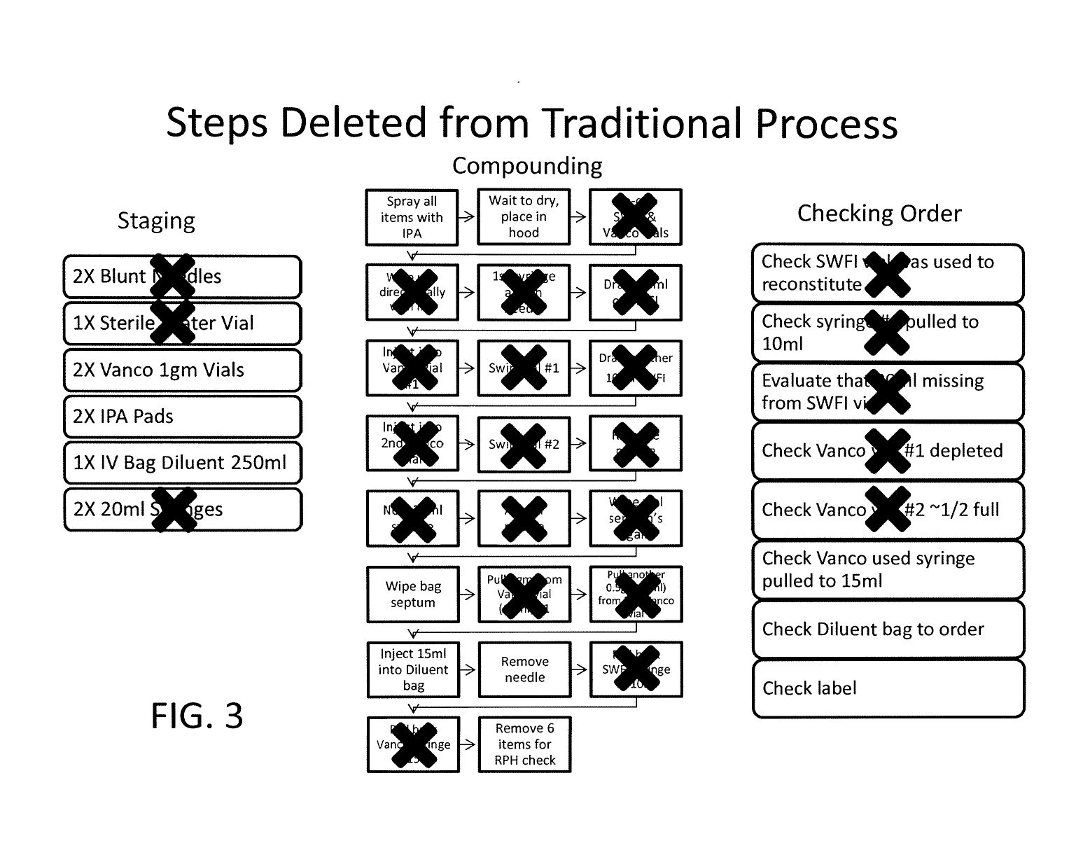

In this way, the point-of-care staging, compounding, and checking process includes significantly fewer steps and raw materials relative to the traditional process, resulting in substantial time savings, substantially less waste, and much higher safety as there are fewer opportunities for human error or breaches of sterility. In addition, the systems and methods of the disclosure are particularly useful where urgent care is needed, but point-of-care compounding is unavailable or cannot be completed without significant delay. FIGS. 2 and 3 illustrate how compounding of vancomycin according to the disclosure eliminates much of the traditional compounding process shown in FIG. 1.

In a first aspect, a method for compounding a therapeutic agent for intravenous administration at a point-of-care facility comprises providing a syringe containing the therapeutic agent dissolved in a carrier or pre-dissolved in a carrier at a dose or concentration that is therapeutically effective for a particular patient but which is not commercially available at the therapeutically effective dose or concentration for the patient or is on the U.S. FDA drug shortage list, disinfecting the syringe and disinfecting a port on a container containing a diluent, connecting the disinfected syringe to the disinfected port, injecting the therapeutic agent dissolved in the carrier or pre-dissolved in the carrier into the diluent via the port, and verifying that the syringe is depleted of the therapeutic agent dissolved in the carrier or pre-dissolved in the carrier. According to this method, the therapeutic agent preferably is not dissolved in the carrier or pre-dissolved in the carrier by drawing the carrier into a first syringe, injecting the carrier from the first syringe into a vial containing the agent and mixing the carrier with the agent to obtain a preparation of the agent, removing the preparation of the agent from the vial, and combining the preparation of the agent with another preparation of the agent, whether done by hand or by an automated compounding machine. According to this method, the therapeutic agent is preferably not dissolved in the carrier or pre-dissolved in the carrier by drawing the carrier into a first syringe, injecting the carrier from the first syringe into a first vial containing the agent and mixing the carrier with the agent to obtain a preparation of the agent, removing the preparation of the agent from the first vial, and injecting the preparation of the agent into a second vial containing the agent and mixing the preparation of the agent with the agent in the second vial in order to obtain a concentrated preparation of the agent, whether done by hand or by an automated compounding machine.

The disinfected syringe may be connected to the disinfected port via any suitable connection. The syringe may comprise a needle or luer lock, with a compatible counterpart structure at the port (e.g., puncturable closure for the needle, or the male or female end of a luer lock). The syringe may comprise a blunt end or needle-less connector that may be connected to the port.

In a second aspect, a method for compounding a therapeutic agent for intravenous administration at a point-of-care facility comprises providing a vial comprising a puncturable closure and containing the therapeutic agent dissolved in a carrier or pre-dissolved in a carrier at a dose or concentration that is therapeutically effective for a particular patient but which is not commercially available at the therapeutically effective dose or concentration for the patient or is on the U.S. FDA drug shortage list, disinfecting the closure and, optionally, disinfecting a port comprising a spike and connected to a container containing a diluent, connecting the vial to the port such that the spike punctures the closure, adding the therapeutic agent dissolved in the carrier or pre-dissolved in the carrier to the diluent through the port, and verifying that the vial is depleted of the therapeutic agent dissolved in the carrier or pre-dissolved in the carrier. According to this method, the therapeutic agent preferably is not dissolved in the carrier or pre-dissolved in the carrier by drawing the carrier into a syringe, injecting the carrier from the syringe into the vial containing the agent and mixing the carrier with the agent to obtain a preparation of the agent, whether done by hand or by an automated compounding machine. According to this method, the therapeutic agent preferably is not dissolved in the carrier or pre-dissolved in the carrier by drawing the carrier into a first syringe, injecting the carrier from the syringe into the vial containing the agent and mixing the carrier with the agent to obtain a preparation of the agent, removing the preparation of the agent from the vial, and combining the preparation of the agent with another preparation of the agent, or injecting the preparation of the agent into a second vial containing the agent in powder form and mixing the preparation of the agent with the agent in a second vial in order to obtain a concentrated preparation of the agent, whether done by hand or by an automated compounding machine. According to this method, it is preferred that no diluent is removed from the container prior to allowing the therapeutic agent dissolved in the carrier or pre-dissolved in the container to flow into the diluent via the port, and it is preferred that the volume of the agent dissolved in the carrier or pre-dissolved in the carrier is less than 10% of the volume of the diluent in the container.

The port comprising a spike may be connected to the container by the manufacturer, such that the port and the container are an integral unit. In the alternative, the port comprising a spike may be a separate structure that is compatible with and connected by hand to an inlet/medication port on the container. The MINI-BAG plus container system by Baxter Healthcare Corp. is a non-limiting example of a suitable integral port-container unit.

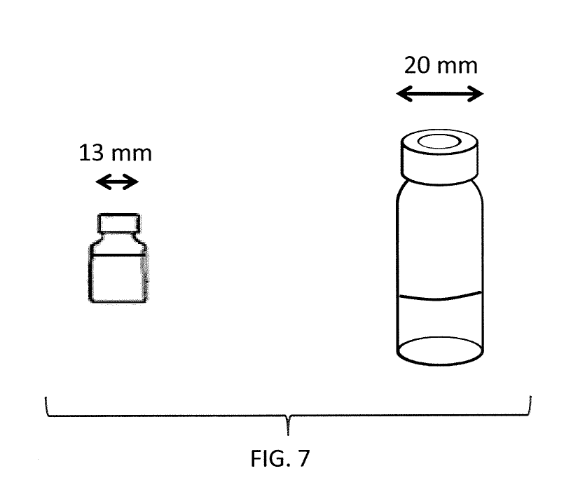

The vial preferably includes a significant volume of empty space, such that the therapeutic agent dissolved in the carrier or pre-dissolved in the carrier comprises a ratio of the carrier volume to the empty space of the vial of about 1 (carrier volume) to about 1 (empty space volume) or greater. The vial preferably includes a significant volume of empty space, such that the therapeutic agent dissolved in the carrier or pre-dissolved in the carrier comprises a ratio of the carrier volume to the empty space of the vial of about 1 (carrier volume) to at least about 1 (empty space volume) or greater. The ratio of carrier volume to empty space may be about 1 to about 2 or greater, about 1 to about 3 or greater, about 1 to about 4 or greater, about 1 to about 5 or greater, about 1 to about 10 or greater, or about 1 to about 20 or greater. The closure of the vial preferably is about 20 mm in diameter (FIG. 7). In some aspects, the vial preferably is not more than hall-full, and is preferably less than half-full. By way of example, but not of limitation, a vial having a 20 ml liquid capacity will not include more than 10 ml of liquid, reconstituted therapeutic agent.

In a third aspect, a method for compounding a therapeutic agent for intravenous administration at a point-of-care facility comprises providing a vial comprising a puncturable closure and containing the therapeutic agent dissolved in a carrier or pre-dissolved in a carrier at a dose or concentration that is therapeutically effective for a particular patient but not commercially available at the therapeutically effective dose or concentration for the patient or is on the U.S. FDA drug shortage list, disinfecting the closure and disinfecting a port comprising a spike, connecting the vial to the disinfected port such that the spike punctures the closure, and connecting the port to a container containing a diluent, adding the therapeutic agent dissolved in the carrier or pre-dissolved in the carrier to the diluent through the port, and verifying that the vial is depleted of the therapeutic agent dissolved in the carrier. According to this method, the therapeutic agent preferably is not dissolved in the carrier or pre-dissolved in the carrier by drawing the carrier into a syringe, injecting the carrier from the syringe into the vial containing the agent and mixing the carrier with the agent to obtain a preparation of the agent, whether done by hand or by an automated compounding machine. According to this method, the therapeutic agent preferably is not dissolved in the carrier or pre-dissolved in the carrier by drawing the carrier into a first syringe, injecting the carrier from the syringe into the vial containing the agent and mixing the carrier with the agent to obtain a preparation of the agent, removing the preparation of the agent from the vial, and combining the preparation of the agent with another preparation of the agent, whether done by hand or by an automated compounding machine. According to this method, no diluent is removed from the container prior to allowing the therapeutic agent dissolved in the carrier or pre-dissolved in the carrier to flow into the diluent via the port. According to this method, the volume of the agent dissolved in the carrier or pre-dissolved in the carrier is less than 10% of the volume of the diluent in the container.

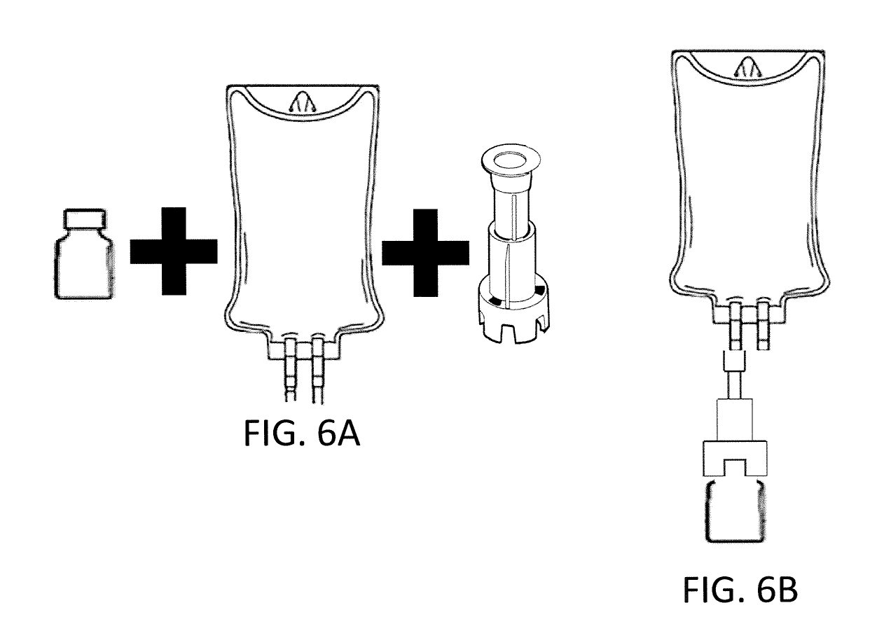

The port comprising a spike may be separate from the container, though the port is compatible with an inlet/medication port on the container (FIG. 6B). The user connects the port comprising a spike to the inlet/medication port on the container. The VIAL MATE port by Baxter Healthcare Corp. is a non-limiting example of a suitable port for mating with an inlet/medication port on a diluent container.

The vial preferably includes a significant volume of empty space, such that the therapeutic agent dissolved in the carrier or pre-dissolved in the carrier comprises a ratio of the carrier volume to the empty space of the vial of about 1 (carrier volume) to about 1 (empty space volume) or greater. The vial preferably includes a significant volume of empty space, such that the therapeutic agent dissolved in the carrier or pre-dissolved in the carrier comprises a ratio of the carrier volume to the empty space of the vial of about 1 (carrier volume) to at least about 1 (empty space volume) or greater. The ratio of carrier volume to empty space may be about 1 to about 2 or greater, about 1 to about 3 or greater, about 1 to about 4 or greater, about 1 to about 5 or greater, about 1 to about 10 or greater, or about 1 to about 20 or greater. The closure of the vial may be 13 mm in diameter. More preferably the closure is about 20 mm in diameter.

In any of the methods described or exemplified herein, the carrier in which the therapeutic agent is dissolved may be any suitable pharmaceutically acceptable carrier. The carrier may be hydrophobic or aqueous. Non-limiting examples of suitable carriers include water (e.g., sterile water for injection), physiologic saline, physiologic dextrose, aqueous alcohols such as ethanol, aqueous acetic acid, and other suitable carriers. The carrier may further comprise one or more of a buffer, preservative, antimicrobial agent, or stabilizer.

In any of the methods described or exemplified herein, the dose or concentration may vary, for example, according to the particular therapeutic agent as well as its commercially available amount. The dose or concentration may also vary according to the characteristics and/or condition of the patient.

In any of the methods described or exemplified herein, the container is a vessel used for intravenous infusions, and typically is a bag but may also be a bottle or other suitable container. The container includes a diluent into which the agent-containing carrier is mixed. In the syringe embodiment, the agent-containing carrier is mixed with the diluent by injecting the agent-containing carrier into the diluent container, for example, via a port connected to the container.

In any of the methods described or exemplified herein, the carrier and therapeutic agent are preferably pre-mixed together (for example, as therapeutic agent pre-dissolved in a carrier). The agent is obtained in its approved/distributed amount, with its container disinfected using appropriate Current Good Manufacturing Practice (CGMP) production methods, governed by the US FDA. The therapeutic agent in solid form (powder, crystals, etc.) is combined with additional amounts of the therapeutic agent in solid form until the desired amount for the preparation is reached, with the combined amount weighed to verify the desired amount is attained, and other additives such as buffers, excipients, preservatives, and other suitable additives may be combined with the therapeutic agent in solid form. The desired amount of the agent in solid form (+/−additives) is then added to and mixed together with the carrier, for example, by using peristaltic pumps to feed liquid carrier in and to agitate or sufficiently solubilize the agent within the carrier. The therapeutic agent in liquid form is combined with additional volume of the agent in liquid form until the desired volume and concentration for the preparation is reached, and other additives such as buffers, excipients, preservatives, and other suitable additives may be combined with the therapeutic agent in liquid form. Following mixing, the solution is sterilized by filtration or other sterilization methodology according to USP <71>. The sterilized solution is then packaged at the desired volume into a syringe or vial. The solution may be stored at room temperature, frozen, or under refrigeration until use, when it is mixed together with the intravenous diluent according to a method for compounding a therapeutic agent for intravenous administration as described or exemplified herein.

The therapeutic agent may be pre-dissolved in a carrier in a way that also streamlines the manufacturing process. As a non-limiting example, morphine may be pre-dissolved in a carrier as a master batch at 50 mg/ml (not available commercially), and apportioned as 1 ml, 2 ml, or 3 ml to make morphine 50 mg/1 ml, 100 mg/2 ml, and 150 mg/3 ml. To prepare syringes or vials at 0.1 mg/ml, a master batch of morphine at 100 mg/2 ml is injected into a 1 liter bag of a diluent, then apportioned via automation or robotics to make one thousand doses of the morphine at 0.1 mg/ml (1 ml prepackaged in a 3 ml syringe).

Referring to FIG. 2, the staging process 210 includes obtaining a syringe 212, a disinfectant 214, and a diluent 216. After the staging process 210 is complete the compounding process 220 commences. In the compounding process 220 the disinfecting step 222 comprises placing the disinfectant 214 in contacted with the syringe 212 and diluent 216. If the disinfectant 214 is a liquid (e.g., 70% isopropyl alcohol), the drying step 224 entails permitting the syringe 212, and diluent 216 to become dry or substantially dry. The wiping step 226 follows the drying step 224. In the wiping step 226, excess disinfectant 214 is removed from the diluent 216 (e.g., the diluent bag's septum is wiped). In the following injection step 228, the syringe 212 is placed in contact with the diluent 216 and the contents of the syringe 212 are injected into the diluent 216. Next, in the withdrawal step 230, the syringe 212 is removed from contact with the diluent 216. Finally, in the removal step 232, the syringe and diluent are provided to a registered pharmacist for completion of the checking process 240. The checking process 240 may also be referred to as a “verification process”. During the checking process, the registered pharmacist reviews at least: (1) the syringe 212 to confirm it is 100% depleted; (2) the final diluent 216 against the patient's order; and (3) the labels of all relevant pharmaceuticals.

Some point-of-care facilities use robots to fill syringes and IV bags. Thus, the systems may be used in accordance with such robots. By way of example, the point-of-care facility may take five individual vials of commercially available morphine (1 mg/ml in 20 ml vials). The robot draws the content of each vial (total 100 ml) and then injects the 100 ml volume into an empty IV bag. The robot will then add 900 ml of diluent to the bag to bring the total volume to 1 liter as a stock solution; no sterility/potency/endotoxin testing is being done on this stock solution since this is internal use. The concentration of the stock solution is 0.1 mg/ml, and takes about 2 to 3 hours to prepare. Then, this solution is apportioned into one thousand syringes solely for internal use. Such robots are slow, filling approximately 40 syringes per hour, such that the robot will have to work about 25 hours to fill one thousand syringes. Thus, the morphine being pre-dissolved in a carrier saves about 27 hrs of production time at the point-of-care facility.

In any of the methods described or exemplified herein, the diluent may be any solution suitable for intravenous administration. The diluent may comprise a hypotonic, isotonic, or hypertonic solution. The diluent may comprise a colloid solution or a cystalloid solution. Non-limiting examples of suitable diluents include sodium chloride, for example, at 0.9%, 0.25%, 0.45%, or 0.7%, dextrose, for example, at 2.5%, or Lactated Ringer's (LR) solution, D5W, D5NS, or D5LR solution, and normosol solution.

In any of the methods described or exemplified herein, the disinfecting steps may comprise wiping, spraying, or immersing the component (e.g., port, syringe, needle, or connections thereof) with a disinfectant. The disinfectant may be any suitable material used in compounding or aseptic techniques according to USP <797>, including sterile isopropyl alcohol or peroxide.

The therapeutic agent may be any agent that is capable of intravenous administration, and which may be compounded or repackaged to attain a dose or concentration that is therapeutically effective for a particular patient. The therapeutic agent may include any newly invented agent that is to be intravenously administered, as well as any existing agents that are later formulated for intravenous administration.

Non-limiting examples of suitable categories of therapeutic agents include one or more of calcium channel blockers, all chemical classes of antibiotics, local anesthetics, contrast agents, epidural agents, electrolyte supplement, analgesic (e.g., opioid, non-steroidal anti-inflammatory), corticosteroids, proton pump inhibitors, h2 antagonists, anticholinergics, vasopressors (e.g., sympathomimetics & catecholamines), anticoagulants, benzodiazepines, oxytocic agents, alpha-adrenergic receptor agents, beta-adrenergic blocking agents, antimicrobials, and anticonvulsants.

Non-limiting examples of the therapeutic agent (including salt form) include one or more of hydromorphone, midazolam, morphine, norepinephrine, oxytocin, phenylephrine, ropivacaine, bupivacaine, lidocaine, vancomycin, gentamicin, atropine, betamethasone, calcium gluconate, cefazolin, dexamethasone, epinephrine, ephedrine, esmolol, fentanyl, flumazeril, glycopyrrolate, heparin, hydralazine, ketamine, labetalol, magnesium sulfate, metropropolol, caffeine injectable, rocuronium, sodium citrate, sodium thiosulfate, xanthine, succinylcholine, vasopressin, verapamil, or vecuronium.

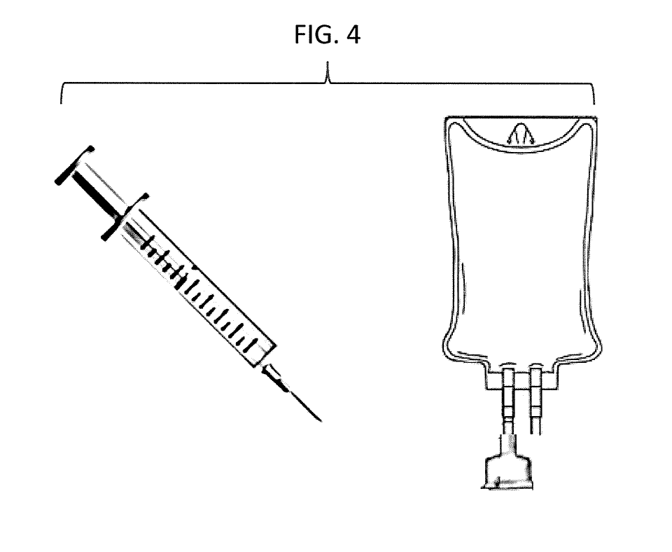

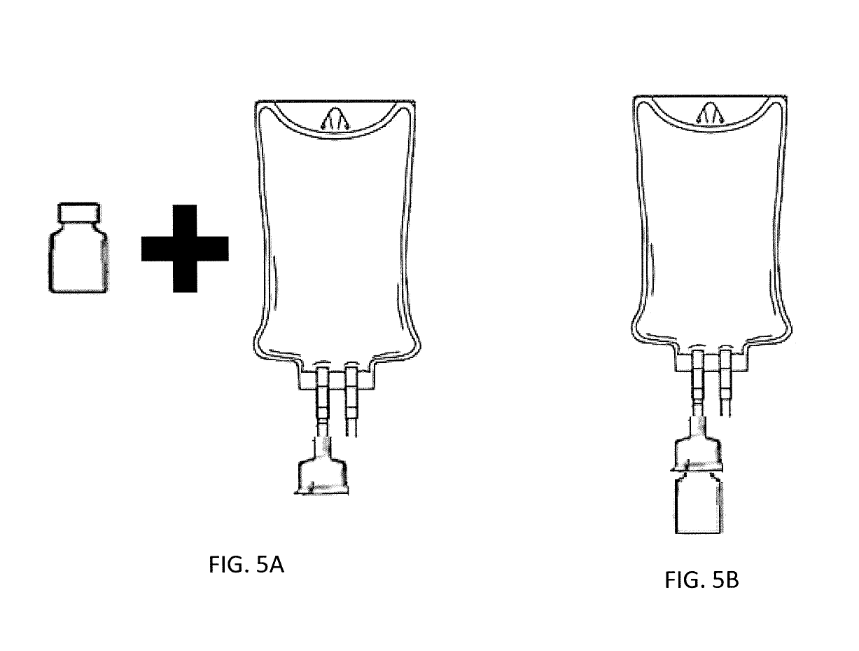

Systems for practicing the methods described herein include a container comprising a diluent for intravenous administration and a syringe (FIG. 4) or a vial (FIGS. 5A, 5B, 6A, 6B, and 7) containing a therapeutic agent dissolved in a carrier at a dose or concentration that is therapeutically effective for a particular patient but not commercially available at the therapeutically effective dose or concentration. In some aspects, the container comprises a port comprising a spike, which port is integral with the container (FIG. 5A). In some aspects, the container comprises a port comprising a spike, though the port is a separate component that is capable of being connected to the container (FIG. 6A).

Although illustrated and described above with reference to certain specific embodiments and examples, the present invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges. It is also expressly intended that the steps of the methods of using the various devices disclosed above are not restricted to any particular order.

What is claimed is:

1. A method for compounding a therapeutic agent for intravenous administration, comprising:
providing a syringe containing the therapeutic agent dissolved in a carrier at a dose or concentration that is therapeutically effective for a particular patient but is not commercially available at the therapeutically effective dose or concentration or is on the U.S. FDA drug shortage list, disinfecting the syringe and disinfecting a port on a container containing a diluent, connecting the disinfected syringe to the disinfected port, injecting the therapeutic agent dissolved in the carrier into the diluent via the port, and verifying that the syringe is depleted of the therapeutic agent dissolved in the carrier;
provided that the therapeutic agent is not dissolved in the carrier by drawing the carrier into a first syringe, injecting the carrier from the first syringe into a vial containing the agent in powder form and mixing the carrier with the agent to obtain a reconstituted preparation of the agent, removing the reconstituted preparation of the agent from the vial, and combining the reconstituted preparation of the agent with another reconstituted preparation of the agent.

2. The method according to claim 1, wherein the therapeutic agent is a calcium channel blocker, antibiotic, contrast agent, local anesthetic, epidural agent, electrolyte supplement, opioid analgesic, non-steroidal anti-inflammatory analgesic, corticosteroid, proton pump inhibitor, h2 antagonist, anticholinergic, sympathomimetic vasopressor, catecholamine vasopressor, anticoagulant, benzodiazepine, oxytocic agent, alpha-adrenergic receptor agent, beta-adrenergic blocking agent, or anticonvulsant.

3. The method according to claim 1, wherein the therapeutic agent comprises one or more of hydromorphone, midazolam, morphine, norepinephrine, oxytocin, phenylephrine, ropivacaine, bupivacaine, lidocaine, vancomycin, gentamicin, atropine, betamethasone, calcium gluconate, cefazolin, dexamethasone, diltiazem, epinephrine, ephedrine, esmolol, fentanyl, flumazeril, glycopyrrolate, heparin, hydralazine, ketamine, labetalol, magnesium sulfate, metropropolol, xanthine, caffeine, rocuronium, sodium citrate, sodium thiosulfate, succinylcholine, vasopressin, verapamil, or vecuronium.

4. The method according to claim 1, wherein the carrier comprises water and, optionally, one or more buffers.

5. The method according to claim 1, wherein the diluent is an isotonic fluid, a hypotonic fluid, or a hypertonic fluid.

6. The method according to claim 1, wherein the method further comprises connecting the disinfected syringe to the disinfected port via a needle.

7. A method for compounding a therapeutic agent for intravenous administration, comprising:
providing a vial comprising a puncturable closure and containing the therapeutic agent dissolved in a carrier at a dose or concentration that is therapeutically effective for a particular patient but is not commercially available at the therapeutically effective dose or concentration or is on the U.S. FDA drug shortage list, disinfecting the closure and optionally disinfecting a port comprising a spike and connected to a container containing a diluent, connecting the vial to the port such that the spike punctures the closure, adding the therapeutic agent dissolved in the carrier to the diluent through the port, and verifying that the vial is depleted of the therapeutic agent dissolved in the carrier;
provided that the therapeutic agent is not dissolved in the carrier by drawing the carrier into a syringe, injecting the carrier from the syringe into the vial containing the agent in powder form and mixing the carrier with the agent to obtain a reconstituted preparation of the agent, removing the reconstituted preparation of the agent from the vial, and combining the reconstituted preparation of the agent with another reconstituted preparation of the agent, provided that no diluent is removed from the container prior to allowing the therapeutic agent dissolved in the carrier to flow into the diluent via the port, and provided that the volume of the agent dissolved in the carrier is less than 10% of the volume of the diluent in the container.

8. The method according to claim 7, wherein the therapeutic agent is a calcium channel blocker, antibiotic, contrast agent, local anesthetic, epidural agent, electrolyte supplement, opioid analgesic, non-steroidal anti-inflammatory analgesic, corticosteroid, proton pump inhibitor, h2 antagonist, anticholinergic, sympathomimetic vasopressor, catecholamine vasopressor, anticoagulant, benzodiazepine, oxytocic agent, alpha-adrenergic receptor agent, beta-adrenergic blocking agent, or anticonvulsant.

9. The method according to claim 7, wherein the therapeutic agent comprises one or more of hydromorphone, midazolam, morphine, norepinephrine, oxytocin, phenylephrine, ropivacaine, bupivacaine, lidocaine, vancomycin, gentamicin, atropine, betamethasone, calcium gluconate, cefazolin, dexamethasone, diltiazem, epinephrine, ephedrine, esmolol, fentanyl, flumazeril, glycopyrrolate, heparin, hydralazine, ketamine, labetalol, magnesium sulfate, metropropolol, xanthine, caffeine, rocuronium, sodium citrate, sodium thiosulfate, succinylcholine, vasopressin, verapamil, or vecuronium.

10. The method according to claim 7, wherein the carrier comprises water and, optionally, one or more buffers.

11. The method according to claim 7, wherein the diluent is an isotonic fluid a hypotonic fluid, or a hypertonic fluid.

12. The method according to claim 7, wherein the vial containing the therapeutic agent dissolved in the carrier comprises a ratio of carrier volume to empty space of about 1 to about 1 or greater.

13. The method according to claim 7, wherein the vial containing the therapeutic agent dissolved in the carrier comprises a ratio of carrier volume to empty space of about 1 to about 2 or greater.

14. A method for compounding a therapeutic agent for intravenous administration, comprising:
providing a vial comprising a puncturable closure and containing the therapeutic agent dissolved in a carrier at a dose or concentration that is therapeutically effective for a particular patient but is not commercially available at the therapeutically effective dose or concentration or is on the U.S. FDA drug shortage list, disinfecting the closure and disinfecting a port comprising a spike, connecting the vial to the disinfected port such that the spike punctures the closure, and connecting the port to a container containing a diluent, adding the therapeutic agent dissolved in the carrier to the diluent through the port, and verifying that the vial is depleted of the therapeutic agent dissolved in the carrier;
provided that the therapeutic agent is not dissolved in the carrier by drawing the carrier into a syringe, injecting the carrier from the syringe into the vial containing the agent in powder form and mixing the carrier with the agent to obtain a reconstituted preparation of the agent, removing the reconstituted preparation of the agent from the vial, and combining the reconstituted preparation of the agent with another reconstituted preparation of the agent, provided that no diluent is removed from the container prior to allowing the therapeutic agent dissolved in the carrier to flow into the diluent via the port, and provided that the volume of the agent dissolved in the carrier is less than 10% of the volume of the diluent in the container.

15. The method according to claim 14, wherein the therapeutic agent is a calcium channel blocker, antibiotic, contrast agent, local anesthetic, epidural agent, electrolyte supplement, opioid analgesic, non-steroidal anti-inflammatory analgesic, corticosteroid, proton pump inhibitor, h2 antagonist, anticholinergic, sympathomimetic vasopressor, catecholamine vasopressor, anticoagulant, benzodiazepine, oxytocic agent, alpha-adrenergic receptor agent, beta-adrenergic blocking agent, or anticonvulsant.

16. The method according to claim 14, wherein the therapeutic agent comprises one or more of hydromorphone, midazolam, morphine, norepinephrine, oxytocin, phenylephrine, ropivacaine, bupivacaine, lidocaine, vancomycin, gentamicin, atropine, betamethasone, calcium gluconate, cefazolin, dexamethasone, diltiazem, epinephrine, ephedrine, esmolol, fentanyl, flumazeril, glycopyrrolate, heparin, hydralazine, ketamine, labetalol, magnesium sulfate, metropropolol, xanthine, caffeine, rocuronium, sodium citrate, sodium thiosulfate, succinylcholine, vasopressin, verapamil, or vecuronium.

17. The method according to claim 14, wherein the carrier comprises water and, optionally, one or more buffers.

18. The method according to claim 14, wherein the diluent is an isotonic fluid, a hypotonic fluid, or a hypertonic fluid.

19. The method according to claim 14, wherein the vial containing the therapeutic agent dissolved in the carrier comprises a ratio of carrier volume to empty space of about 1 to about 1 or greater.

20. The method according to claim 14, wherein the vial containing the therapeutic agent dissolved in the carrier comprises a ratio of carrier volume to empty space of about 1 to about 2 or greater.
